(19) United States
(12) Patent Application Publication
(10) Pub. No.: US 2007/0049964 A1
(43) Pub. Date: Mar. 1, 2007

(54) Intraluminal filter for distal protection

(75) Inventors: Albert Dunfee, Byfield, MA (US); Barbara DeVaux, Haverhill, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/214,632

(22) Filed: Aug. 30, 2005

(51) Int. Cl. A61M 29/00

(52) U.S. Cl. 606/200

(57) ABSTRACT

The present invention is an intraluminal filtering device for collecting debris in a body lumen. The filter includes a proximal filter portion for receiving debris attached to a distal filter portion for collecting debris. The proximal filter portion includes a plurality of non-overlapping components that are joined together to form openings that allow the passage of embolic debris into an interior of the filter. The proximal filter portion is preferably cylindrically shaped in a compressed state and substantially tapered shaped in an uncompressed state. The distal filter portion is formed by braiding wires and is joined at a proximal end to the proximal filter portion, such as by spot welding or soldering. The filtering device is self-expanding and is adaptable to be positioned and expanded or collapsed by a push-pull mechanism or sheath assembly.

### FIELD OF THE INVENTION

The invention relates generally to intraluminal distal protection devices for capturing particulate in the vessels of a patient. More particularly, the invention relates to a filter for capturing emboli in a blood vessel during an interventional vascular procedure.

### BACKGROUND OF THE INVENTION

Catheters have long been used for the treatment of diseases of the cardiovascular system, such as treatment or removal of stenosis. For example, in a percutaneous transluminal coronary angioplasty (PTCA) procedure, a catheter is used to insert a balloon into a patient's cardiovascular system, position the balloon at a desired treatment location, inflate the balloon, and remove the balloon from the patient. Another example is the placement of a prosthetic stent that is placed in the body on a permanent or semi-permanent basis to support weakened or diseased vascular walls to avoid closure or rupture thereof.

These non-surgical interventional procedures often avoid the necessity of major surgical operations. However, one common problem associated with these procedures is the potential release of embolic debris into the bloodstream that can occlude distal vasculature and cause significant health problems to the patient. For example, during deployment of a stent, it is possible for the metal struts of the stent to cut into the stenosis and shear off pieces of plaque which become embolic debris that can travel downstream and lodge somewhere in the patient's vascular system. Further, pieces of plaque material can sometimes dislodge from the stenosis during a balloon angioplasty procedure and become released into the bloodstream.

Medical devices have been developed to attempt to deal with the problem created when debris or fragments enter the circulatory system during vessel treatment. One technique includes the placement of a filter or trap downstream from the treatment site to capture embolic debris before it reaches the smaller blood vessels downstream. The placement of a filter in the patient's vasculature during treatment of the vascular lesion can collect embolic debris in the bloodstream.

It is known to attach an expandable filter to a distal end of a guidewire or guidewire-like member that allows the filtering device to be placed in the patient's vasculature. The guidewire allows the physician to steer the filter to a downstream location from the area of treatment. Once the guidewire is in proper position in the vasculature, the embolic filter can be deployed to capture embolic debris. Some embolic filtering devices utilize a restraining sheath to maintain the expandable filter in its collapsed configuration. Once the proximal end of the restraining sheath is retracted by the physician, the expandable filter will transform into its fully expanded configuration. The restraining sheath can then be removed from the guidewire allowing the guidewire to be used by the physician to deliver interventional devices, such as a balloon angioplasty catheter or a stent delivery catheter, into the area of treatment. After the interventional procedure is completed, a recovery sheath can be delivered over the guidewire using over-the-wire techniques to collapse the expanded filter (with the trapped embolic debris) for removal from the patient's vasculature. Both the delivery sheath and recovery sheath should be relatively flexible to track over the guide wire and to avoid straightening the body vessel once in place.

Another distal protection device known in the art includes a filter mounted on a distal portion of a hollow guidewire or tube. A moveable core wire is used to open and close the filter. The filter is coupled at a proximal end to the tube and at a distal end to the core wire. Pulling on the core wire while pushing on the tube draws the ends of the filter toward each other, causing the filter framework between the ends to expand outward into contact with the vessel wall. Filter mesh material is mounted to the filter framework. To collapse the filter, the procedure is reversed, i.e., pulling the tube proximally while pushing the core wire distally to force the filter ends apart. A sheath catheter may be used as a retrieval catheter at the end of the interventional procedure to reduce the profile of the “push-pull” filter, as due to the embolic particles collected, the filter may still be in a somewhat expanded state. The retrieval catheter may be used to further collapse the filter and smooth the profile thereof, so that the filter guidewire may pass through the treatment area without disturbing any stents or otherwise interfering with the treated vessel.

However, regardless of how a distal protection filter is expanded during a procedure, i.e., sheath delivered or by use of a push-pull mechanism, a crossing profile of the collapsed filter is to be at a minimum to reduce interference between the filter and other interventional devices or in-placed stents. As well, a compact filter profile is beneficial in crossing severely narrowed areas of vascular stenosis. Thus, what is needed is a filter that achieves a compact, reduced profile without sacrificing the strength and stability needed for effective embolic capture and retention.

### BRIEF SUMMARY OF THE INVENTION

The present invention is an intraluminal filtering device for collecting debris in a body lumen. The filter includes a proximal filter portion for receiving debris attached to a distal filter portion for collecting debris. In an embodiment, the proximal filter portion includes a plurality of non-overlapping components that are joined together to shape openings that allow the passage of embolic debris into an interior of the filter. In another embodiment, the proximal filter portion is an unbraided, one-piece structure with openings for embolic debris formed therein.

The proximal filter portion is preferably cylindrically shaped in a compressed state and includes a body portion that has a substantially frusto-conical or tapered shape in an uncompressed state. In an embodiment, the distal filter portion is a braided filter formed by braiding wires. In another embodiment, the distal filter portion is of a suitable mesh or porous material that collects embolic debris while permitting fluid to flow there through, such as blood flow sufficient for perfusion of body tissues. The distal filter portion is joined at a proximal end to the proximal filter portion, such as by spot welding or soldering. The filtering device is preferably self-expanding and is adaptable to be expanded or collapsed by a push-pull mechanism or sheath assembly.

The present invention also includes a method of making an embolic filter by providing a proximal filter portion of a single material thickness and a braided distal filter portion, and joining the filter portions by spot welding or soldering.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is an illustration of a filter system in accordance with an embodiment of the present invention deployed within a blood vessel.

FIG. 2 is an illustration of a filter system in accordance with an embodiment of the present invention deployed within the coronary arterial anatomy.

FIG. 3 is a side view of a distal protection device in accordance with the present invention.

FIG. 4 is a proximal end view of the distal protection device of FIG. 3 along line A-A.

FIG. 5 is a side view of another embodiment of a distal protection device in accordance with the present invention.

### DETAILED DESCRIPTION OF THE INVENTION

Specific embodiments of the present invention are now described with reference to the figures, where like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

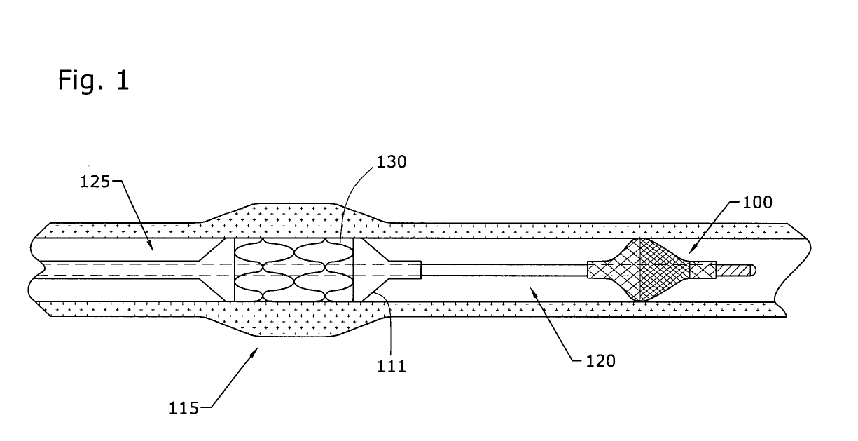

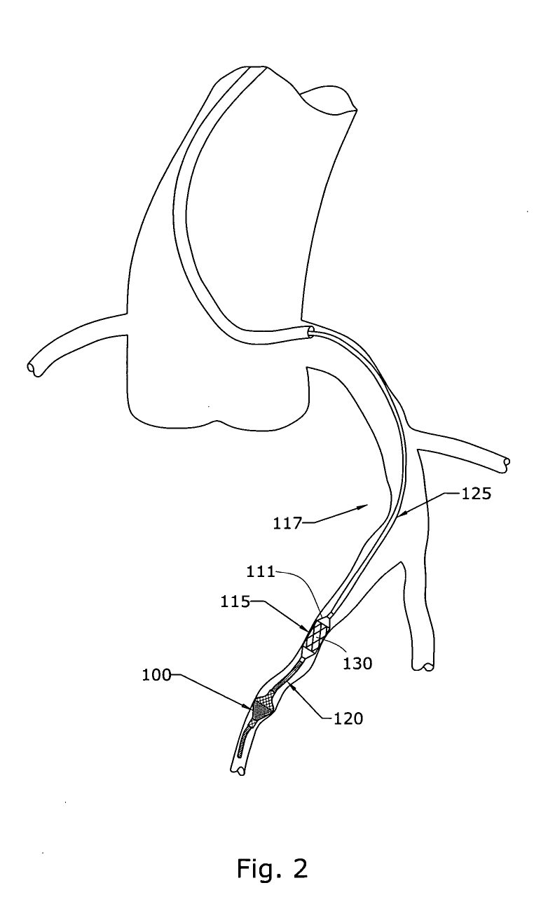

The present invention is a temporary distal protection device for use on a filter guidewire in minimally invasive procedures, such as vascular interventions or other procedures, where the practitioner desires to capture embolic material that may be dislodged during the procedure. With reference to FIGS. 1 and 2, deployment of balloon expandable stent 130 is accomplished by threading catheter 125 through the vascular system of the patient until stent 130 is located within a stenosis at predetermined treatment site 115. Once positioned, balloon 111 of catheter 125 is inflated to expand stent 130 against the vascular wall to maintain the opening. Stent deployment can be performed following treatments such as angioplasty, or during initial balloon dilation of the treatment site, which is referred to as primary stenting.

Catheter 125 is typically guided to treatment site 115 by a guidewire. In cases where the target stenosis is located in tortuous vessels that are remote from the vascular access point, such as with the coronary arteries 117 as shown in FIG. 2, a steerable guidewire is commonly used. According to an embodiment of the present invention, a filter guidewire generally designated as 120 guides catheter 125 to treatment site 115 and includes distally disposed filter 100 to collect embolic debris that may be generated during the procedure.

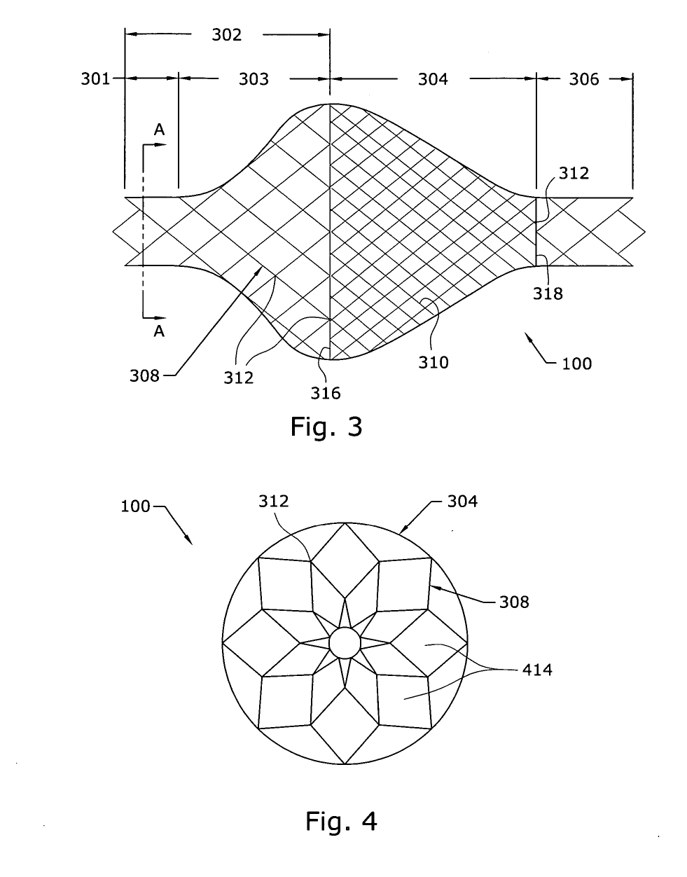

The present invention is directed to a distal protection device, viz., temporary filter 100, which has a reduced profile in its collapsed configuration or state. Filter 100 is preferably secured at least at its proximal and distal ends to filter guidewire 120 in a fixed or slidable arrangement depending on the method of deployment to be utilized. FIGS. 3 and 4 illustrate filter 100 in accordance with an embodiment of the present invention. Filter 100 includes a proximal filter portion 302, a distal filter portion 304 and a distal neck portion 306. Proximal filter portion 302 includes a plurality of proximal components 308, each of which is an independent module or section. In the illustrated embodiment, each proximal component 308 is a ring-like structure formed by a zigzag-shaped wire. In another embodiment, each proximal component 308 may include a sinuous wire of regular or irregular turns joined at its ends to form a ring-like structure. A method for forming various shaped ring-like structures without a joint for use as proximal components in the present invention is disclosed in U.S. Pat. No. 5,292,331, which is incorporated by reference herein in its entirety.

Each proximal component 308 is attached to an adjoining proximal component 308 by at least one non-overlapping joint 312. In an embodiment where proximal components 308 are of stainless steel, nitinol, or a nickel-based super alloy, joint 312 may be achieved by laser welding, spot welding or soldering the components together without overlapping in a radial direction. That is, ring-like proximal components 308 are joined in a serially stacked arrangement along a central axis of filter 100, with each joint 312 being formed between two side-by-side filaments. In this manner, proximal filter portion 302 has no more than a single material thickness, as measured radially throughout its structure and, therefore, is of a reduced profile in its collapsed or compressed configuration. In addition, proximal components 308 are joined one to another in a manner so as to shape and size openings 414 for receiving debris there through. Openings 414 are shown substantially diamond-shaped in the embodiment of FIG. 3, but may be of any other shape that optimizes receipt of embolic debris within filter 100.

Further, proximal filter portion 302 is preferably produced to achieve cylindrical neck portion 301 and substantially frusto-conical or tapered body portion 303 in an uncompressed or expanded configuration or state, in order to properly enlarge and position openings 414 to receive, rather than obstruct, debris in the fluid flow of a body lumen. In an embodiment of the present invention, a proximal filter portion having a desired expanded configuration that is substantially frusto-conical or tapered may be shaped or pre-formed by heat treating proximal components 308 comprised of a shape memory material, such as nitinol, over a suitably shaped mandrel, as disclosed in U.S. Pat. No. 6,325,815 that is incorporated by reference herein in its entirety. In another embodiment, proximal filter portion 302 includes a cylindrical neck portion 301 and a body portion 303 that steps up in outer diameter from the neck portion to where it attaches to distal filter portion 304. The stepped increase may be accomplished by joining ring-like proximal components 308 of increasing outer diameters, serially arranged proximal-to-distal along body portion 303.

Distal filter portion 304 for filtering and collecting embolic debris is attached at proximal end 316 to proximal filter portion 302 and at distal end 318 to neck portion 306. As illustrated in FIG. 3, distal neck portion 306 is comprised of a plurality of modular components, preferably of a metallic material, joined one to another in a manner similar to that described with reference to proximal portion 302. Filter 100 is fixedly or slidably coupled to filter guidewire 120 at neck portion 306 and, therefore, the modular components that form neck portion 306 are not joined to form openings sized for passage of embolic debris.

In the embodiment of FIG. 3, distal filter portion 304 is a braided filter comprised of a plurality of wires or filaments 310 that are woven together to form the tubular braided filter. In an embodiment of the present invention, braiding wires or filaments are preferably made from stainless steel, a shape memory material, such as nitinol, or a nickel-based super alloy. In another embodiment, the distal filter portion may be formed from a suitable mesh or porous material that collects embolic debris while permitting fluid to flow there through, such as blood flow sufficient for perfusion of body tissues. Such mesh filters and braided filters are disclosed in U.S. Pat. No. 6,346,116 that is incorporated by reference herein in its entirety.

Distal filter portion 304 is sized and shaped such that when it is fully deployed, as in FIG. 1, proximal end 316 will contact the inner surface of the blood vessel wall into which it is placed. The inner surface contact is preferably maintained over substantially the entire expanded circumference of proximal end 316 of distal filter portion 304 to prevent any emboli from escaping past filter 100. Alternatively, the contacting surface of filter 100 may be cylindrical, and/or the joint between proximal filter portion 302 and distal filter portion 304 may be disposed on, or located distally or proximally of the contacting surface (not shown).

In the embodiment of FIG. 3, proximal filter portion 302, distal filter portion 304, and distal neck portion 306 are made from the same or compatible, i.e. joinable, metallic materials and are joined to form a unitary member that is filter 100. Accordingly, filter portions 302, 304, 306 are securable to each other by joints 312, e.g., by laser welding, spot welding or soldering, that are strong enough to permanently unite the filter portions. It may be desirable to form all of joints 312 without a radial overlapping of respective portions at their interfaces. In this manner, filter 100 can be made to have a less bunched, reduced profile in its collapsed or compressed configuration.

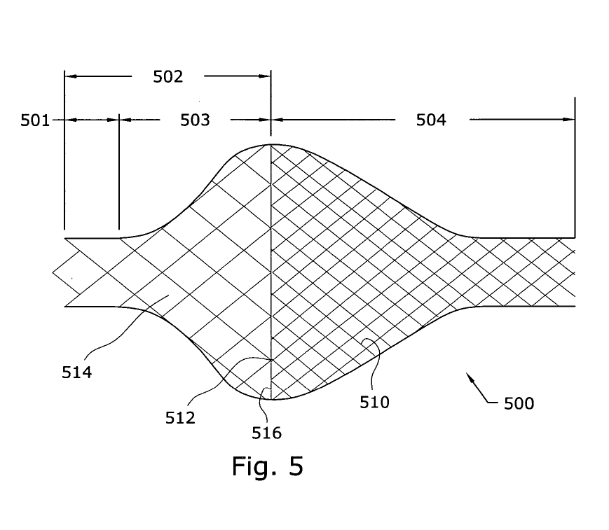

FIG. 5 illustrates another embodiment of the present invention. Embolic filter 500 includes proximal filter portion 502 that is formed as a one-piece, completely integral unit from a thin-walled tube, preferably of a heat treatable material. Proximal filter portion 502 includes cylindrical neck portion 501 with body portion 503 that has openings 514 created therein. Openings 514 are sized, when filter 500 is in an expanded or deployed configuration, to receive debris and may be formed by cutting or machining using, e.g., laser, water jet, electrical discharge machining, or chemical etching techniques. Accordingly, proximal filter portion 502 may be formed without welds or joints assuring a single material thickness throughout its structure and, therefore, a compact profile in its collapsed or compressed configuration. Similar to the embodiment of FIG. 3, proximal filter portion 502 may be produced to achieve a substantially frusto-conical or tapered shape in an uncompressed or expanded configuration, in order to properly enlarge and position openings 514 to receive, rather than obstruct, debris in the fluid flow of a body lumen. A suitable method for forming such a unitary proximal filter portion is disclosed in U.S. Pat. No. 6,613,079 B1, which is incorporated by reference herein in its entirety.

In the embodiment of FIG. 5, distal filter portion 504 is a braided filter that includes a braided neck portion formed integrally therewith. Distal filter portion 504 is attached at proximal end 516 to proximal filter portion 502. In the embodiment of FIG. 5, proximal and distal filter portions 502, 504 are made from the same or compatible (joinable) metallic materials and are joined to form a unitary member, i.e., filter 500. Accordingly, filter portions 502, 504 are securable to each other by joints 512, e.g., by laser welding, spot welding or soldering, that are strong enough to permanently unite the filter portions. It may be desirable to form all of joints 512 without a radial overlapping of respective portions at their interfaces. In this manner, filter 500 may be made to have a less bunched, reduced profile in its collapsed or compressed configuration. Filter 500 is securable to a filter guidewire along its proximal and distal ends to be delivered to and deployed at a treatment site in accordance with the disclosure below.

A filter in accordance with the present invention may be transformable between its collapsed and expanded configurations by relative movement between its ends. Such movement may be accomplished by a filter guidewire mechanism similar to that shown in any of the filter guidewires disclosed in U.S. Pat. No. 6,706,055, U.S. Pat. No. 6,818,006 and U.S. Pat. No. 6,866,677, which are incorporated by reference herein in their entireties. Alternatively, a filter in accordance with the present invention may be deployed and/or retrieved via a sheath catheter, such as by the method and apparatus disclosed in U.S. Pat. No. 6,059,814, which is incorporated by reference herein in its entirety, or the '116 patent previously incorporated by reference. The transformation of the filter may be impelled by external mechanical means alone or by self-shaping memory (either self-expanding or self-collapsing) within the filter. Preferably, filters 100, 500 are self-expanding, meaning that filters 100, 500 have a mechanical memory to return to the expanded, or deployed configuration. As previously discussed, such mechanical memory can be imparted to the metal comprising filters 100, 500 by thermal treatment to achieve a spring temper in stainless steel, for example, or to set a shape memory in a susceptible metal alloy, such as nitinol. In such an embodiment of the present invention, it is preferable that at least the majority of braiding wires 310, 510 forming distal filter portion 304, 504 and proximal components 308 forming proximal filter portion 302 be capable of being heat treated into the desired filter shape/component, and such wires should also have sufficient elastic properties to provide the desired self-expanding or self-collapsing features. In a similar manner, the tube forming proximal filter portion 502 may be heat treated into the desired filter shape, as previously discussed.

Optionally, radiopaque markers (not shown) may be placed on proximal and distal ends of filters 100, 500 to aid in fluoroscopic observation during manipulation thereof. Alternatively, fluoroscopic visualization of filters 100, 500 may be enhanced when at least one of the filaments includes a wire having enhanced radiopacity compared to conventional non-radiopaque wires suitable for braiding distal filter portion 304, 504 or forming proximal components 308. Braiding wire 310, 510 having enhanced radiopacity may be made of, or coated with a radiopaque metal such as gold, platinum, tungsten, alloys thereof, or other biocompatible metals having a relatively high X-ray attenuation coefficient compared with stainless steel or nitinol. One or more filaments having enhanced radiopacity may be inter-woven with non-radiopaque wires, or all wires comprising filters 100, 500 may have the same enhanced radiopacity.

Alternatively, one or more of braiding wires/braid filaments 310, 510 and/or proximal components 308 may comprise a composite wire having a radiopaque core and non-radiopaque layer or casing. Such coaxial, composite wires are referred to as DFT (drawn-filled-tube) wires in the metallic arts, and filters comprising such wires are disclosed in U.S. Pat. No. 6,866,677 B2 that is incorporated by reference herein in its entirety.

While various embodiments of the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A filtering device for collecting debris in a body lumen, comprising:
a proximal filter portion for receiving debris there through, the proximal filter portion having a plurality of non-overlapping components that are conjoined to form openings there between, wherein the proximal filter portion is cylindrically shaped in a compressed state and substantially frusto-conically shaped in an uncompressed state; and
a distal filter portion for collecting debris, the distal filter portion attached to the proximal filter portion.

2. The filtering device of claim 1, wherein the distal filter portion is comprised of wire braid having a porosity sufficient to allow fluid flow there through.

3. The filtering device of claim 2, wherein the proximal and distal filter portions are comprised of the same material.

4. The filtering device of claim 3, wherein the material is selected from the group consisting of stainless steel, nitinol, and nickel-based super alloys.

5. The filtering device of claim 1, wherein a distalmost component is welded to a proximal end of the distal filter portion.

6. The filtering device of claim 1, wherein the distal filter portion is comprised of a mesh material.

7. The filtering device of claim 1, wherein each of the non-overlapping components is a ring-like structure.

8. The filtering device of claim 7, wherein the ring-like structure is comprised of a zigzag-shaped wire that is joined at its ends.

9. The filtering device of claim 7, wherein the proximal and distal filter portions are comprised of the same material.

10. The filtering device of claim 7, wherein a distalmost ring-like structure is welded to a proximal end of the distal filter portion.

11. A filtering device for collecting debris in a body lumen, comprising:
an unbraided proximal filter portion comprised of openings sized to receive debris there through and having a substantially cylindrical configuration in a compressed state and a substantially tapered configuration in an uncompressed state; and
a distal filter portion for collecting debris, the distal filter portion being fixedly attached at a proximal end to a distal end of the proximal filter portion.

12. The filtering device of claim 11, wherein the distal and proximal filter portions have an interface that is substantially non-overlapping in a radial direction.

13. The filtering device of claim 11, wherein the unbraided proximal filter portion is comprised of a plurality of modular components joined one to another without overlapping in a radial direction to form the openings there between.

14. The filtering device of claim 13, wherein each of the modular components is a ring-like structure.

15. The filtering device of claim 14, wherein the ring-like structure is comprised of a zigzag-shaped wire.

16. The filtering device of claim 13, wherein the distal filter portion is comprised of braided wires.

17. The filtering device of claim 11, wherein the unbraided proximal filter portion is a one-piece structure with the openings formed therein.

18. The filtering device of claim 17, wherein the distal filter portion is comprised of braided wires.
